(19) United States
(12) Patent Application Publication
(10) Pub. No.: US 2023/0383224 A1
(43) Pub. Date: Nov. 30, 2023

(54) LAB-ON-CHIP DEVICES FOR SIMULATING FUNCTION AND DISEASE OF A COMBINED NASAL AND LUNG AIRWAY SYSTEM

(71) Applicant: THE BOARD OF TRUSTEES OF THE UNIVERSITY OF ARKANSAS, Little Rock, AR (US)

(72) Inventors: Amanda Walls, Fayetteville, AR (US); Kartik Balachandran, Fayetteville, AR (US); Manon van Vegchel, Fayetteville, AR (US)

(21) Appl. No.: 18/202,075

(22) Filed: May 25, 2023

Related U.S. Application Data
(60) Provisional application No. 63/345,527, filed on May 25, 2022.

(51) Int. Cl. C12M 3/00; C12N 5/071; C12M 1/12; C12M 1/00

(52) U.S. Cl. CPC C12M 21/08; C12N 5/0697; C12M 25/02; C12M 29/08

(57) ABSTRACT

An apparatus for simulating an airway, including: an air channel having a central portion with an air inlet at a first end and an air outlet at a second end opposite the first end; and a vascular channel adjacent to the central portion of the air channel, the vascular channel being separated from an interior of the central portion of the air channel by a porous membrane, the air channel being configured to conduct air from the air inlet through the central portion such that air moves adjacent to the porous membrane.

### CROSS-REFERENCE TO RELATED APPLICATIONS

The present application claims the benefit of priority under 35 U.S.C. § 119(e) to U.S. Provisional Application No. 63/345,527, as filed May 25, 2022, the contents of which are incorporated herein by reference in its entirety.

### STATEMENT REGARDING FEDERALLY SPONSORED RESEARCH

N/A.

### BACKGROUND

Organ- and tissue-on-a-chip models can be used to recreate complex biological systems in vitro, making these systems amenable to repeatable scientific analyses. There is a need for such a model of the respiratory system, particularly in light of the global SARS-CoV-2 virus outbreak and the resulting impact on the respiratory pathway.

### SUMMARY OF THE INVENTION

Accordingly, new systems, methods, and apparatus for simulating an airway are desirable.

Thus, in one embodiment the disclosure provides an apparatus for simulating an airway, including: an air channel having a central portion with an air inlet at a first end and an air outlet at a second end opposite the first end; and a vascular channel adjacent to the central portion of the air channel, the vascular channel being separated from an interior of the central portion of the air channel by a porous membrane, the air channel being configured to conduct air from the air inlet through the central portion such that air moves adjacent to the porous membrane.

In another embodiment the disclosure provides a method for simulating an airway, including: providing an airway simulation apparatus including: an air channel having a central portion with an air inlet at a first end and an air outlet at a second end opposite the first end, and a vascular channel adjacent to the central portion of the air channel, the vascular channel being separated from an interior of the central portion of the air channel by a porous membrane; and conducting, using the air channel, air from the air inlet through the central portion such that air moves adjacent to the porous membrane.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a diagram of a Nasal Airway-Lung-on-Chip (AirLoC) platform according to certain aspects of the disclosure.

FIG. 2A shows a photograph of a nasal portion of an AirLoC platform featuring a “static” version of a media chamber with electrodes for measuring trans-epithelial or trans-endothelial resistance incorporated therein.

FIG. 2B shows a diagram of an AirLoC platform according to some aspects of the disclosure.

FIG. 2C shows a diagram of an AirLoC platform according to various aspects of the disclosure.

FIG. 2D shows a photograph of a nasal portion of an AirLoC platform showing placement of a polycarbonate membrane attached between the nasal portion of the air channel and the nasal vascular channel.

FIG. 3 shows results of computer modeling of a velocity profile of air flow at peak inhalation in the nasal section of the device.

FIG. 4 shows a velocity profile of air flow at a point in the center of the nasal section of an AirLoC device based on computer modeling.

FIG. 5 shows a schematic of a desired cellular arrangement in the AirLoC system.

FIG. 6 shows a fluorescence microscopy image of cultured human nasal epithelial cells (hNECs) stained for cell nuclei (using DAPI dye) on the membrane of the chip construct. The image was taken through the clear wall of the chip, without deconstructing. Cells were fixed for imaging after 7 days in culture on the chip.

FIG. 7 shows a graph of transepithelial electrical resistance (TEER) measurements of hNECs on the polycarbonate membrane over a period of 27 days. The air-liquid interface (ALI) was established on day 7, as indicated in the graph. TEER values peak after ALI is established and stabilized, which is indicative of a tight epithelial barrier formation.

FIG. 8 shows immunocytochemistry images of hNECs after 21 days following establishment of an ALI while cultured on polycarbonate membranes. Images show well-differentiated nasal mucosa with expressed cilia (left) and mucous proteins (right). Left: acetylated-alpha-tubulin (green), ZO-1 (red), DAPI (blue). Right: MUC5AC (green), ZO-1 (red), DAPI (blue).

FIG. 9 shows a diagram of an AirLoC platform according to certain aspects of the disclosure. The left panel shows a perspective view and the right panel shows a side elevation view.

FIG. 10 shows plots of flow rate (Panel A), Reynold's number (Panel B), and Womersley number (Panel C) based on possible chip dimensions. The red boxes in (Panel A) and (Panel C) indicate physiologically relevant values. All values below the red line in (Panel B) are below 2300, which is considered laminar flow. In (Panel C), “bpm” is breaths per minute.

FIGS. 11A-11C depict CFD Simulation results showing velocity through the inlet (FIG. 11A), over the center (FIG. 11B), and across the YZ plane of the nasal chip (FIG. 11C).

FIG. 12 depicts CFD Simulation results showing WSS on the membrane portion of the nasal chip (top), and across the chip surface (bottom).

FIG. 13 shows cells stained for DAPI (blue) and ZO-1 (red) and expressing acetylated-tubulin (green, Panel A) and MUC5AC (green, Panel B). (Panel C) MTS viability assay (p>0.1).

FIG. 14 shows a schematic (top) and photograph (bottom) of a construction of an airflow set up.

FIG. 15 shows ICC images of control cells (Panel A) and flow-exposed cells (Panel B) stained for DAPI (blue) and ZO-1 (red). (Panel C) MTS viability assay (p<0.1).

### DETAILED DESCRIPTION

In accordance with some embodiments of the disclosed subject matter, mechanisms (which can include systems, methods, and apparatus) for simulating an airway are provided.

The field of organ-on-chip technology arose from the convergence of microfabrication and tissue engineering. The devices built using this technology can recapitulate key aspects of human physiology in a repeatable, three-dimensional form. Organ-on-chip systems can recreate features such as complex organ functions, tissue-barrier properties, parenchymal tissue function, and multi-organ interactions. The Mechanobiology and Soft Materials Laboratory at the University of Arkansas has extensive experience developing organ-on-chip devices (e.g. for the blood-brain-barrier and heart) that can be used in biomedical research. Embodiments of the present disclosure (referred to herein as the Nasal Airway-Lung-on-Chip, or AirLoC, system) arose from efforts to expand the scope of the laboratory's systems during the global SARS-CoV-2 outbreak. It was understood that the virus has major effects on the respiratory system, yet there were no benchtop micro-physiological systems that replicated both the upper and lower systems concurrently on the same device. This lack of technology has presented a major gap in the biomedical research field, as there are few benchtop technologies that can be used to study the physiological effects of any particulate matter on the combined upper and lower airways.

In various embodiments, the AirLoC device includes a multi-layered cell culture platform, including connected nasal and lung sections. The platform includes an air channel spanning the nasal and lung sections and a pair of vascular channels associated with the nasal section and the lung section which contain cell culture media, which may be static or flowing. Thin micro- or nano-porous membranes (e.g. with pore sizes as small as 3 nm and as large as 10 μm) separate the air and media in both sections. in certain embodiments, the air channel may be attached to a pump to provide controlled air flow at a physiological breathing rate. In various embodiments the air flow may move into and out of the air channel at regular or irregular intervals (e.g. depending on what type of breathing pattern is being simulated) at a rate of 12-16 cycles per minute, similar to normal adult human breathing rates, although slower or faster rates may be used in order to simulate various subjects or health conditions (e.g. faster breathing rates of up to 60 breaths per minute to simulate breathing of small children or infants). Human nasal epithelial cells and human bronchial/tracheal epithelial cells can be seeded on respective porous membranes in the airway side of the device, while human microvascular endothelial cells can be seeded on the reverse side of each membrane. Other cell types found in the airways, such as fibroblasts, can also be cultured within the device. Accordingly, the cellular organization within the AirLoC system can mimic the basic functional unit of the human nasal and lung airways.

Embodiments of the AirLoC device have been designed so that cells cultured on the membranes experience physiological fluid flow both in the airway and in the vascular channels. Accordingly, parameters such as Reynolds (Re=UL/v), Strouhal (St=fL/U), and Womersley (α=(ωL/v)^{1/2}) numbers are used to determine the appropriate channel geometries, air volumes, and flow profiles necessary for the device to simulate human breathing mechanics (where: U—flow velocity; L—channel dimension; v—kinematic viscosity of fluid; f—frequency/periodicity of flow waveform; and ω—angular frequency of flow waveform). In the airway channel, the air flow pump can deliver particulate matter (ranging from nano- to micro-meter diameter scale) and other inhaled species to the system, such as viral particles or corticosteroid aerosol droplets. The distribution pattern of introduced particles is similar to that observed in vivo, thus making the AirLoC device a useful tool in studying the effects of airborne particulate matter on the human respiratory system.

Currently, there are no other existing benchtop microphysiological platforms (i.e. organ-on-chip systems) that mimic the physiology and function of the combined upper and lower respiratory systems. The development of the AirLoC device generates an understanding of combined responses of the upper and lower respiratory airways. Additionally, the AirLoC device can serve as a platform for the discovery of potential new therapeutics for respiratory diseases exacerbated by airborne particulate matter. The data generated by using the device will provide a foundational understanding of the cytotoxicity and physiological effects that result from particulate matter deposition in the airways.

In various embodiments, the AirLoC system may also be employed for other toxicological evaluations. For example, airway effects of ambient air from varying geographic locations could be tested on the platform, as well as airway effects of viral exposure. The use of human cells to engineer the AirLoC also is an advantage over preclinical animal-based approaches. In certain embodiments, the AirLoC can be adapted to custom-build patient-specific devices with various disease phenotypes for the purpose of drug safety and efficacy testing (i.e. to provide personalized medicine).

In various embodiments the AirLoC device is a multilayered cell culture platform including interconnected nasal and lung sections, both of which have airway and vascular channels. An air pump controls air flow through the airway channel, while a peristaltic pump controls media flow through the media channels.

Thus, in one embodiment the disclosure provides an apparatus for simulating a nasal and lung airway which includes an air channel including an extended nasal portion with an air inlet at a first end and a lung portion at a second end opposite the first end. A nasal vascular channel is disposed adjacent to the nasal portion of the air channel, where the nasal vascular channel is separated from an interior of the nasal portion of the air channel by a nasal porous membrane. A lung vascular channel is disposed adjacent to the lung portion of the air channel, where the lung vascular channel is separated from an interior of the lung portion of the air channel by a lung porous membrane. The air channel is configured to conduct air from the air inlet through the nasal portion to the lung portion.

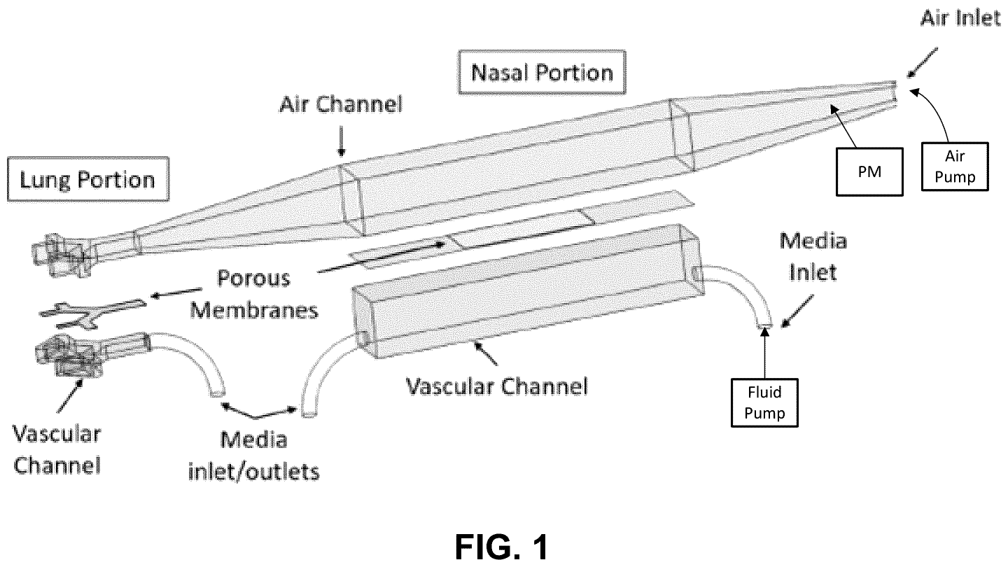

In some embodiments the lung portion of the air channel may include at least one branched passageway for air flow, as shown in FIG. 1. In addition, the lung vascular channel and the lung porous membrane may each include at least one branched portion which is complementary to the at least one branched passageway of the lung portion of the air channel (FIG. 1), mimicking the branched airways found in the distal lung. In certain embodiments, the lung portion of the air channel may be made of a flexible material that mimics the elasticity of the lung and the degree of elasticity can be varied to simulate different disease states (see below). In addition, in some embodiments the nasal portion can also be made of a flexible material to mimic certain physiological conditions.

Also as shown in FIG. 1, a central portion of the nasal portion of the air channel may be enlarged and the first end of the air channel may taper down from the central portion to the air inlet and the second end of the air channel may taper down from the central portion to the lung portion.

In some embodiments, each of the nasal vascular channel and the lung vascular channel may include an inlet and an outlet for flow of cell culture media through the respective channel, although as noted above certain “static” embodiments of the channels may not include inlets for media flow or the inlets may be covered or plugged.

In some embodiments, each of the nasal porous membrane and the lung porous membrane may include a nano-porous or micro-porous membrane, which may be a polycarbonate membrane. In various embodiments the membrane may be made of other materials including polymers such as polytetrafluoroethylene (PFTE) or polyester (PET) or a polyester such as polyethylene terephthalate (PET), where the particular choice of material and specifications (e.g. thickness, pore size, flexibility) will depend on the application.

In various embodiments the air channel and/or vascular channels include cells growing on one or more surfaces thereof, including on one or both of the nasal porous membrane or the lung porous membrane. In some embodiments, a first side of the nasal porous membrane facing the air channel may include nasal epithelial cells growing thereon, and a second side of the nasal porous membrane opposite the first side and facing the nasal vascular channel may include microvascular endothelial cells growing thereon. In other embodiments, a first side of the lung porous membrane facing the air channel may include at least one of bronchial or tracheal epithelial cells growing thereon, and a second side of the lung porous membrane opposite the first side and facing the lung vascular channel may include microvascular endothelial cells growing thereon. In some embodiments, the apparatus may include fibroblasts growing on at least one of the nasal porous membrane or the lung porous membrane. Other cell types may also be grown on the device, as discussed further below.

In some embodiments, electrodes may be used to measure a potential within the apparatus, for example across the layers of epithelial cells that form on the membranes. Thus, in one embodiment, a first electrode may be disposed within the air channel and in electrical communication with the nasal porous membrane, and a second electrode may be disposed within the nasal vascular channel and in electrical communication with the nasal porous membrane. The first electrode and the second electrode may be coupled to an instrument such as an electrophysiological amplifier and configured to obtain at least one of a trans-epithelial electrical resistance or a trans-endothelial electrical resistance (TEER) across the nasal porous membrane. One or both of the electrodes may be in electrical communication with the membrane by touching a portion of an electrically conductive liquid (e.g. cell culture media) which has continuity with the membrane.

In various embodiments, the apparatus may include an air pump coupled to the air channel (FIG. 1), where the air pump may be configured to provide at least one of positive or negative air pressure to create continuous or cyclic air flow within the air channel. For example, the pump may be configured to provide air flow that cycles at a rate of 12-16 times per minute to mimic the rate of air flow in the human lung during breathing, although higher or lower rates may be used particularly to simulate certain conditions such as hyperventilation. In some embodiments, the pump may be configured to distribute particulate matter (e.g. virus particles or steroid droplets) through the air channel, for example by spraying the particulates into the airflow path while the pump is directing air into the air channel (see FIG. 1, box labeled “PM”) or by introducing the particulates into the air pump.

In certain embodiments, the apparatus pay include a liquid pump (e.g. a peristaltic pump, see FIG. 1) coupled to at least one of the nasal vascular channel or the lung vascular channel. The liquid pump may be configured to deliver cell culture media to at least one of the nasal vascular channel or the lung vascular channel.

Nasal Section

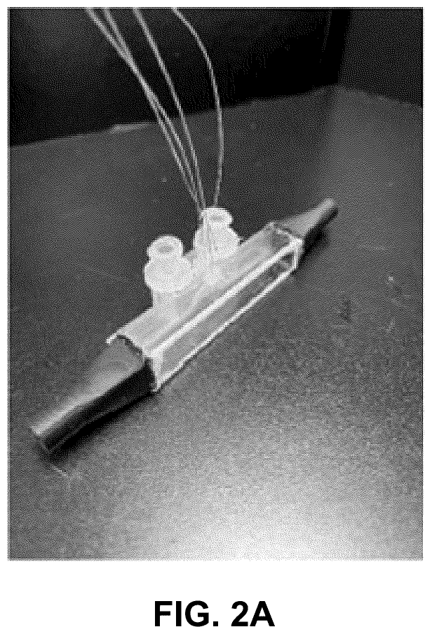

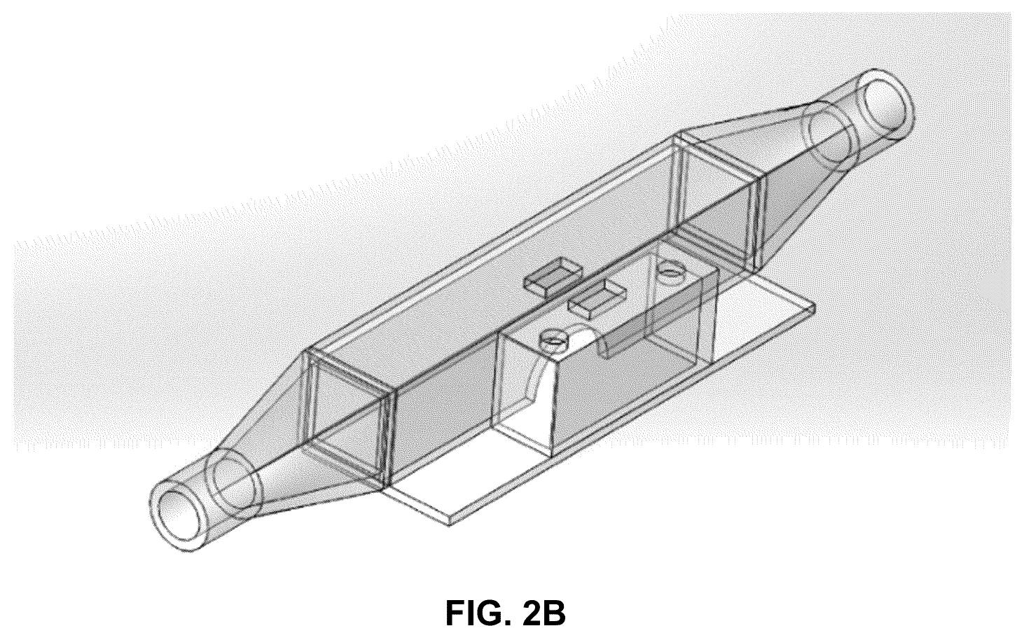

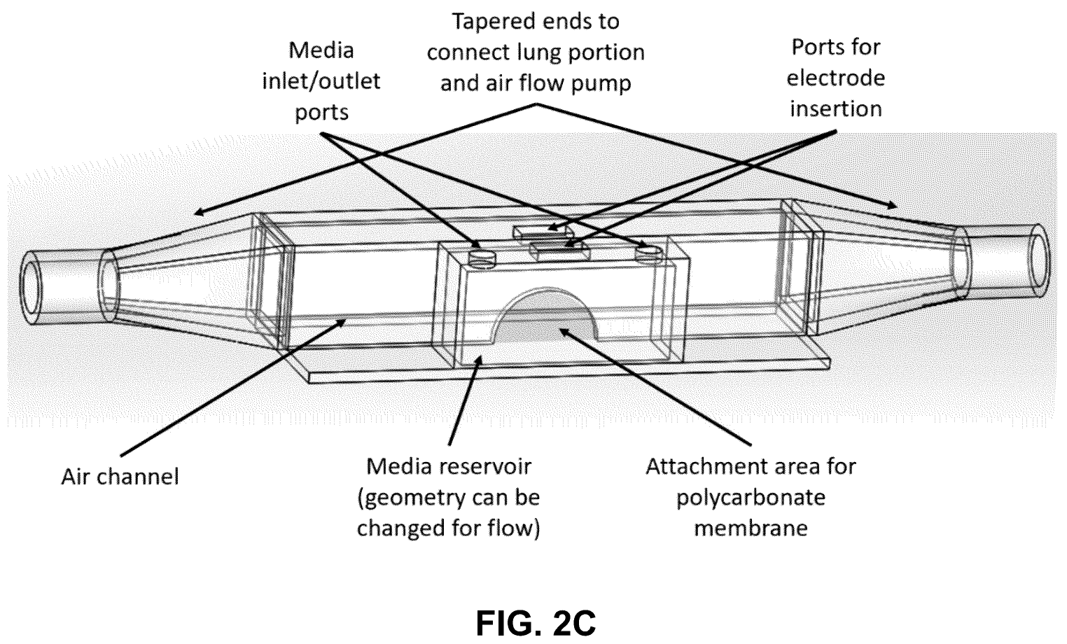

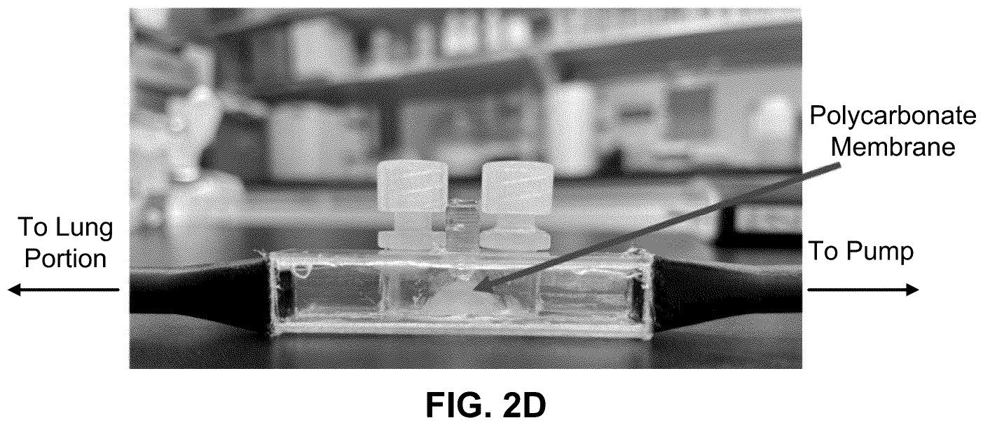

The nasal portion of the airway may be a hollow rectangular prism (although other shapes such as a circular or oval cross-section are also possible) made from materials such as polymethyl methacrylate (PMMA) and polystyrene and may have inner dimensions of 8 mm×8 mm×50 mm and the walls of the channel may be 1 mm thick. Centered along the interior face is an opening where the membrane is bonded (see FIGS. 2A-2D). The porous membrane, which is attached to the well, serves as a well-like area to enclose extracellular matrices (ECM) for epithelial cell growth.

The inlet and outlet of the nasal airway channel may taper to small openings (e.g. using separate ABS plastic pieces bonded to each end, see FIG. 2D) to connect to the pump (inlet) and the lung portion (outlet). The porous membrane may be cut from commercially available thin, porous polycarbonate (PC) or polyethylene terephthalate (PET) sheets and bonded to the opening of the airway channel to enclose the ECM well. A separate channel for media flow with inner dimensions of 8 mm (W)×2 mm (D)×50 mm (L) may also be milled from PMMA. The vascular channel also has an open face at the interior face where it aligns with the porous membrane side of the airway channel. The vascular channel and airway channels may be bonded together around their openings, with the porous membrane sandwiched in between. Inlet and outlet holes may be milled into the vascular channel at either end so that silicone tubing can be inserted and connected to a peristaltic pump. Cap fittings can also be used at the openings of a smaller media channel for static cultures. The pump may be run at flow rate of 36 mL/min to achieve a fluid shear stress of 1 dyn/cm² on the endothelial cells. Nasal epithelial cells can be seeded in the airway channel, while microvascular endothelial cells can be seeded in the vascular channel. The cells can be viewed through the polystyrene base or polystyrene side by a microscope. There are also two small openings, filled with clear silicone, that allow silver electrodes to be inserted for measuring trans-epithelial/endothelial electrical resistance (TEER).

Lung Section

In various embodiments, the end of the nasal airway channel may be tapered to increase the flow rate so that the flow rate of air reaching the bronchial/tracheal airway section is at a physiological rate. In some embodiments, the bronchial airway portion has two levels of branching or bifurcation (although further levels of bifurcation could be incorporated), which mimics the anatomical structure of the trachea and bronchi in the lung. In particular embodiments this branching airway structure is made using soft photolithography techniques to create a master mold, after which polydimethylsiloxane (PDMS) is then poured over the master mold and cured to produce flexible, optically clear channels. A porous PDMS membrane is then fabricated and bonded beneath the bronchial airway channels to form the vascular channel, which may be fabricated using PDMS with the same branching pattern as the airway channel. The airway and vascular channels are bonded together using PDMS, with the porous membrane sandwiched in between. The flexible PDMS allows for the branches to deform at a physiological level and rate as air is pulled into and out of the airway channels. The vascular channels have an inlet and outlet hole where silicone tubing can be inserted and connected to a peristaltic pump to circulate media through the vascular channel. In certain “static” embodiments in which no media is circulated in the lung vascular channel, the inlet and outlet holes may either be plugged up or eliminated altogether.

Previous airway organ- or lab-on-a-chip models have not included the connected nasal and lung portions as disclosed herein. Embodiments of the disclosed design provide an advantage of recapitulating the physiological breathing and subsequent particle distribution for the upper and lower respiratory airways. Furthermore, the AirLoC system uses human cells and thus can reduce the need for animal models/testing while providing relevant data.

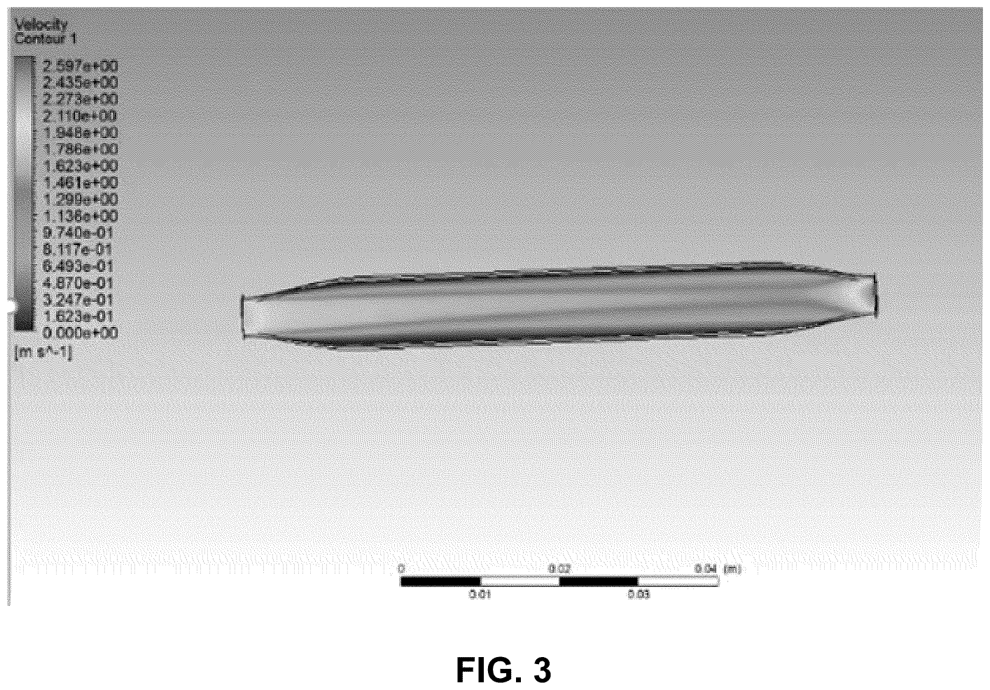

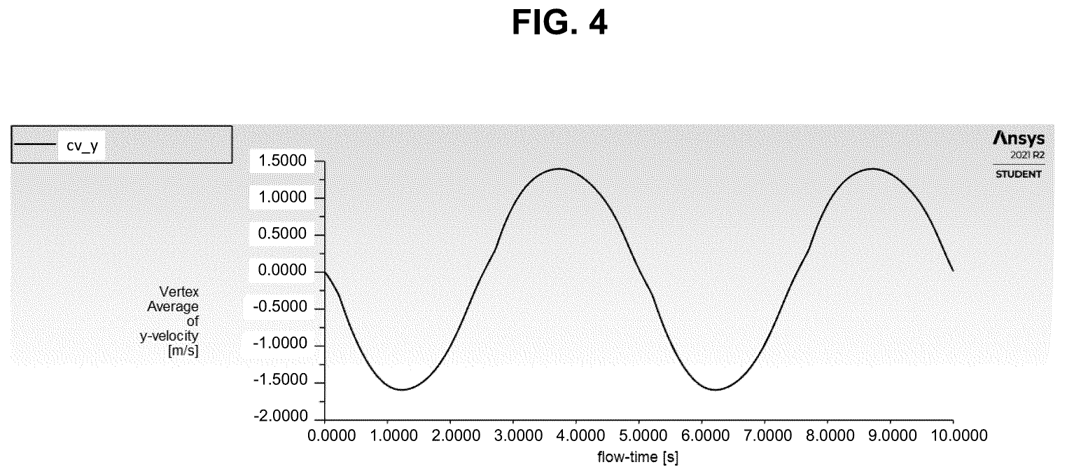

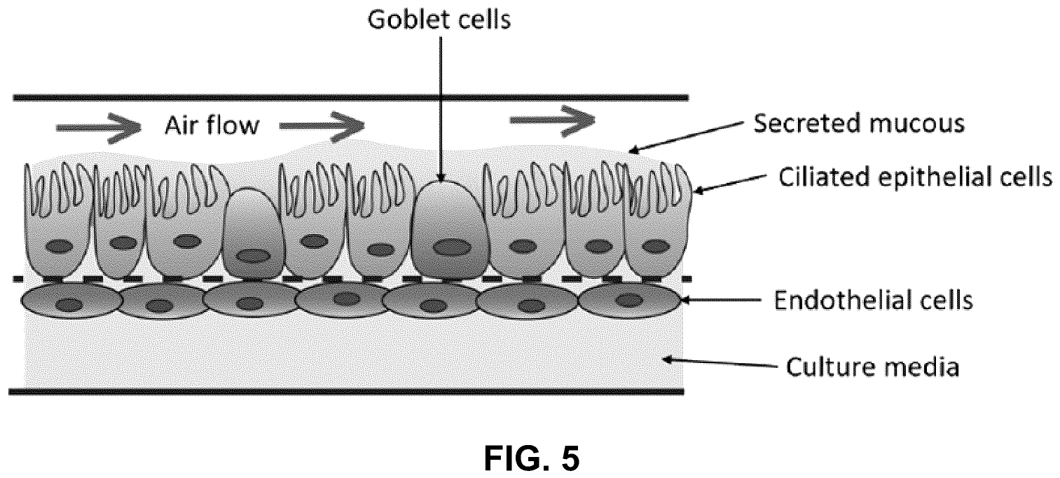

Prototypes of the nasal section of AirLoC have been fabricated and tested with cultured cells to validate cell viability, phenotype, and function. FIG. 5 shows a diagram of anticipated cellular structure, air flow, and culture media distribution within embodiments of the AirLoC platform. Additionally, a computational fluid dynamic (CFD) model (see FIGS. 3, 4) of the nasal portion has been developed to validate that airflow is at physiological breathing rates and patterns. FIG. 3 shows computer modeling of a velocity profile of air flow at peak inhalation in the nasal section of the device. FIG. 4 shows a velocity profile of air flow at a point in the center of the nasal section of an AirLoC device based on computer modeling. Additional testing is being performed to determine the distribution of fluorescent aerosolized particles (1-5 μm diameter) in the chip to validate deposition patterns against computation simulations.

Once the AirLoC system has been fully validated, further studies will be performed to determine the effect of particulate matter inhalation on normal and diseased nasal and lung epithelia, including both healthy cell phenotypes as well as an asthma phenotype. Healthy and diseased AirLoCs will be exposed to various sizes and concentrations of PM-like particles and observed for cytotoxic effects.

Results from these validation studies will contribute to the development of novel in vitro benchtop tools for the study of diseases that affect the upper and lower respiratory systems. Various embodiments of the AirLoC platform can be used to engineer patient-specific nasal airway and lung systems. The platform can also be utilized to study the pathological effects of other airborne pathogens such as viruses on the upper and lower respiratory systems.

Particulate matter (PM) exposure represents a significant risk factor for patients suffering from respiratory illnesses. Unfortunately, effective benchtop “humanized” models that can model PM exposure and their resulting pathological effects on the nasal and lung airways systems do not exist. Lack of these models significantly hinder efforts toward developing therapies for PM exposure-related pathologies.

Accordingly, the disclosed system provides a combined nasal airway and lung-chip platform which mimics the breathing mechanics and air-liquid interface (ALI) of the nasal and lung epithelium. Embodiments of the platform will also include capillary blood flow to mimic transport across the ALI.

PM exposure and drug treatment will be tested on embodiments of the AirLoC platform and the platform will help with understanding disease mechanisms and developing therapeutics for people who suffer from respiratory diseases following PM exposure. In certain embodiments, the AirLoC system can be used to test the effects of particulate matter (smoke, fuel fumes, dust), viral infections (SARS-CoV-2) on nasal and lung function/disease as well as intra-nasal drug delivery testing applications and development of patient-specific nasal/lung airway systems for the purposes of personalized medicine.

Alternate Designs

The following is a description of several alternative embodiments of the AirLoC system:

Different cell types: Several combinations of respiratory cells can be cultured on the device. Instead of, or in addition to, cell types that have been disclosed herein, other examples are as follows:
a. The nasal portion could have olfactory epithelial cells (air side) and neurons (media side) and the lung portion could have bronchial or tracheal epithelial cells (air side) and endothelial cells (media side) to study how toxic PM affects the brain.
b. Fibroblasts could be added to the media side of either portion to study pulmonary fibrosis due to PM exposure.
c. Oral mucosal cells could be used in place of nasal cells in the nasal portion (along with varied air flow parameters) to simulate PM exposure through the mouth.
d. Diseased cells (such as cells with an asthma, rhinitis, or COPD phenotype) could be used in place of healthy cells to study the exacerbated effects of PM.

Change material to model disease: The material stiffness can be altered to model disease states. The lung portion could be made stiffer to simulate fibrotic scar tissue in the lungs or changes in lung mechanical properties with age. The nasal portion could be made flexible to mimic micro-scale expansions/contractions of the nasal tissue during breathing.

Air flow profile/channel dimensions: The dimensions of the device and air flow patterns can be adjusted to model several different aspects of the respiratory passage. Some examples are as follows:
a. Turbulent flow—turbulent flow occurs in particular locations within the respiratory airways, during heavy breathing, or with disease. This can be simulated by changing the chip dimensions and air flow profile (based on non-dimensional numbers analysis).
b. Child vs. Adult breathing: Channel dimensions and air flow patterns can also be altered to simulate child and adult airways.
c. Disease: Often, disease in the airways can cause airway restriction due to inflammation, mucous, polyps, etc. The channel dimensions can be altered to model restrictions and resulting altered air flow patterns.
d. Different locations: The described device specifically models the largest part of the nasal cavity and the large bronchial branches of the lung; however, other locations can be modeled. For example, the smallest bronchi (more branches or less branches), the alveoli, the trachea, larynx, or pharynx.
e. Upper and lower respiratory tract connection: The connection between the upper and lower respiratory tract can be altered (based on non-dimensional numbers and particle deposition equations) to simulate the connecting environment under different states listed above (disease, age, etc.).
f. Physiological breathing vs. positive pressure: The air flow pattern can be changed to simulate physiological breathing, or positive pressure can be applied to simulate exposure to biPAP, CPAP, oxygen cannulas, etc.
g. Particulate Matter Exposure: Air flow patterns and channel dimensions can also be changed to simulate exposure to different types of particulate matter, including nasal sprays, corticosteroid sprays, intranasal vaccines, pollen, exhaust fumes, dust, fungi, etc.

Media channel: The media channel of the device can incorporate flow or be a static reservoir for media. The flow can be connected or separate between both nasal and lung sections. If static, the media reservoir can have caps on openings to seal in media (as shown in diagrams above).

TEER: As disclosed herein, the AirLoC system includes the ability to measure transepithelial electrical resistance (TEER). The electrodes that are needed for making these measurements can be integrated into the device in a number of ways: electrode wires can be fabricated into the device, ports can be included which are compatible with commercially available TEER chopstick electrodes, or a micro-electrode array can be added to the membrane.

Extracellular Matrix: Cells can be seeded in varying ECM environments to produce more two-dimensional (2D) or more three-dimensional (3D) cultures. ECM hydrogels can be added on top of the membrane for a 3D environment.

Disease Phenotypes: Disease phenotypes can be induced on cells by adding different drugs, chemokines, cytokines, etc. These may induce disease such as asthma or rhinitis.

Membrane: The membrane separating the channels of the device does not have to be a specific geometry. It can be altered for a larger/smaller surface area depending on what is desired (i.e. how many cells should be seeded).

Device Coating: Chemical coatings can be applied to the channels within the device to help simulate PM capture or deposition and reduce electrostatic effects.

### EXAMPLES

The following are non-limiting examples of embodiments of the disclosure.

The following are examples of images and other data obtained cells grown on a membrane of an AirLoC system. Among other things, the images and data depict cultured cells that have established an air-liquid interface (ALI) and which have also established a transepithelial electrical resistance (TEER). These Examples demonstrate that the AirLoC system facilitates the culture of cells which functionally and morphologically replicate cells of the human airway and therefore indicate that this system can be used to study the normal and disturbed or diseased state of this organ.

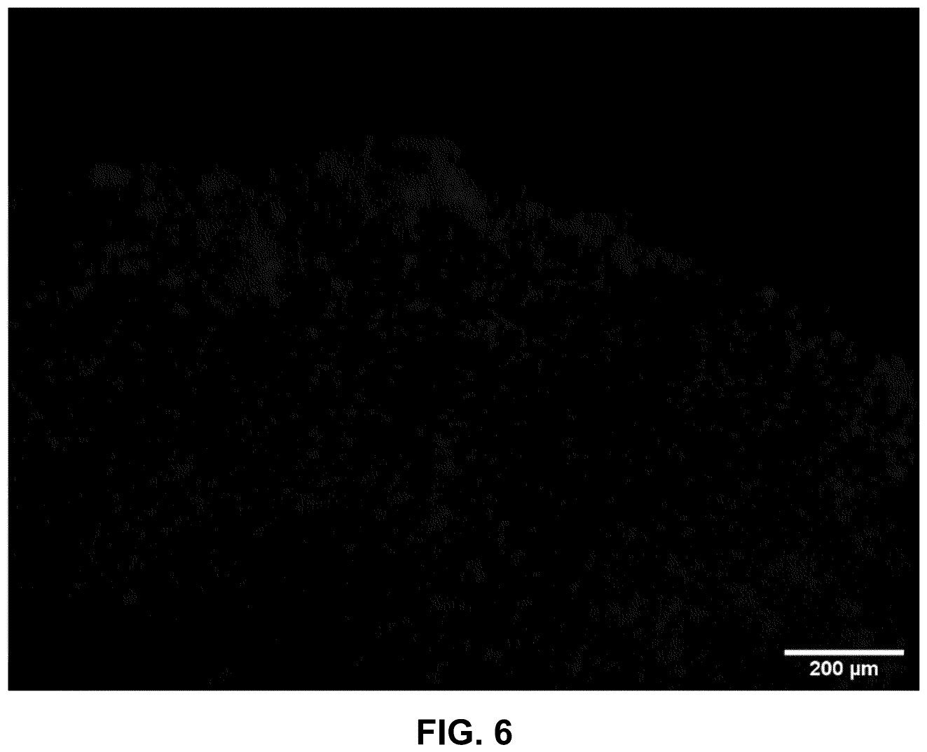

FIG. 6 shows a fluorescence microscopy image of cultured human nasal epithelial cells (hNECs) stained for cell nuclei (using DAPI dye) on the membrane of the chip construct. The image was taken through the clear wall of the chip, without deconstructing. Cells were fixed for imaging after 7 days in culture on the chip. The scale bar in the lower right corner is 200 μm.

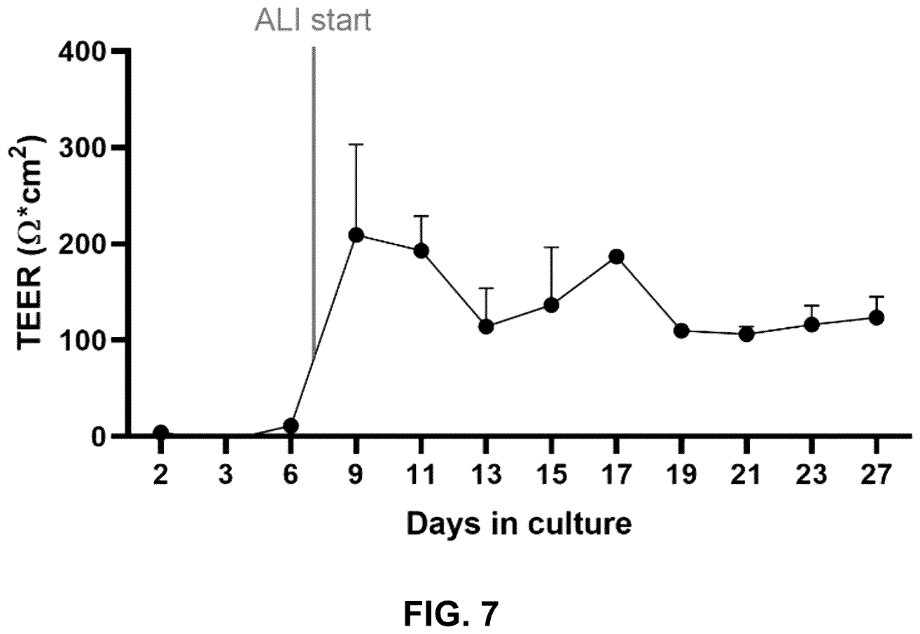

FIG. 7 shows a graph of transepithelial electrical resistance (TEER) measurements of hNECs on the polycarbonate membrane over a period of 27 days. The air-liquid interface (ALI) was established on day 7, as indicated in the graph. TEER values peak after ALI is established and stabilized, which is indicative of a tight epithelial barrier formation.

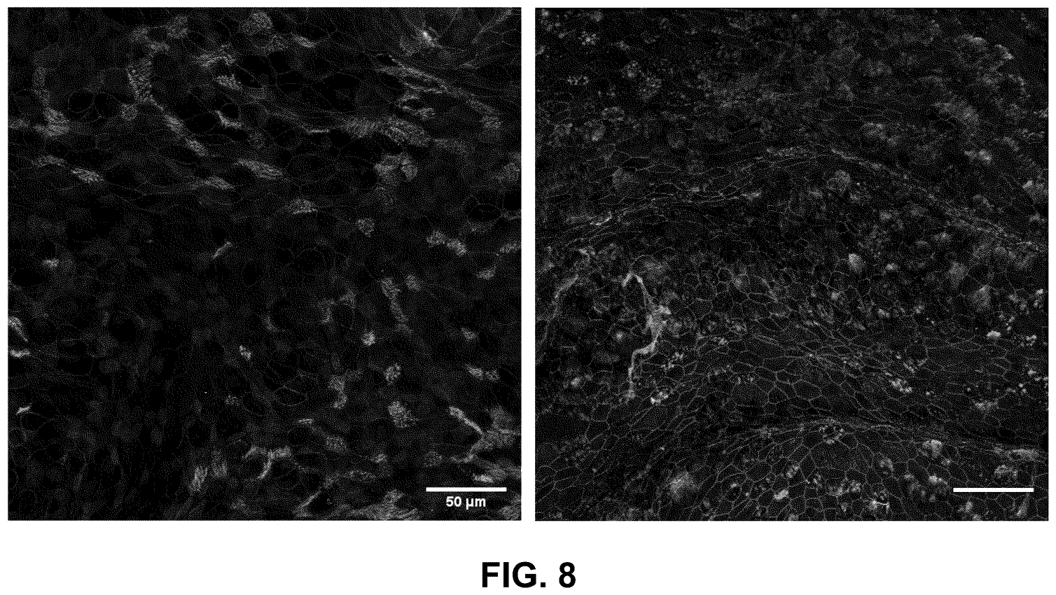

FIG. 8 shows immunocytochemistry images of hNECs after 21 days following establishment of an ALI while cultured on polycarbonate membranes. Images show well-differentiated nasal mucosa with expressed cilia (left) and mucous proteins (right). Left: acetylated-alpha-tubulin (green), ZO-1 (red), DAPI (blue). Right: MUC5AC (green), ZO-1 (red), DAPI (blue).

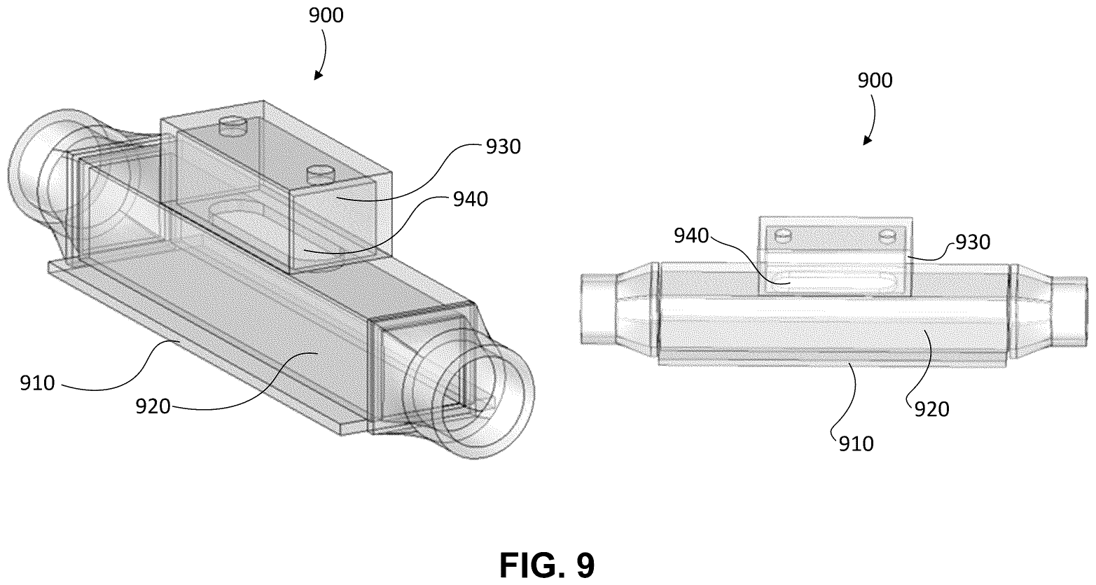

FIG. 9 shows a diagram of an AirLoC platform according to certain aspects of the disclosure; although not shown, the AirLoC platform embodiment of FIG. 9 may be used as a standalone device as shown in FIG. 9 and/or may be used in conjunction with an attached lung portion such as that shown in FIG. 1. Relative to certain other embodiments disclosed herein, the embodiment of FIG. 9 includes tapered ends that are shortened (e.g., relative to the embodiment of FIG. 2, and in some embodiments shortened to 11 mm in length) and the openings widened (e.g., relative to the embodiment of FIG. 2, and in some embodiments widened to 7.5 mm diameter) so that a 1000 μl pipette tip can be inserted into the airflow chamber, for example to facilitate cell culture access and other capabilities.

The AirLoC platform embodiment of FIG. 9 demonstrates an orientation and organization which may provide for one or more of a decreased fabrication time or a reduction in a number of bonded pieces, which in turn may reduce leaking at the various joints. In one embodiment, the AirLoC 900 may include three basic parts including a baseplate 910 (e.g., made of clear polystyrene), an air chamber/air channel 920, and a media chamber/vascular channel 930, which may be stacked onto one another as shown. As with other embodiments, cells may be cultured on a porous membrane disposed in an opening 940 between the air chamber/air channel 920 and the media chamber/vascular channel 930.

Validation by Non-dimensional Analyses and CFD Simulations

The dimensions for the nasal portion of the chip can be optimized based on non-dimensional numbers. From literature, it was found that the three most important numbers used to characterize air flow through the nasal passageways were Reynold's, Womersley, and Strouhal numbers shown in Table 1. The Reynold's number is a function of velocity, chip diameter, and air viscosity and defines the flow as being either laminar or turbulent; the target number was less than 2300 to ensure a laminar flow profile. The Womersley number is a function of chip diameter, breathing frequency, and air viscosity and describes the pulsatile nature of the flow; the target value was between 1.0 and 1.68. The Strouhal number is a function of chip diameter, breathing frequency, and air velocity, and describes the oscillatory or steady nature of the airflow; the target value was less than 1. Since the Reynold's, Womersley, and Strouhal numbers all have common variables, the equations were used to create curves so that the chip dimensions could be selected to satisfy the requirements of the non-dimensional numbers, as well as maintain a physiological level for a resting breathing rate (12-16 cycles/minute) and flow rate (˜200-300 mL/s). All calculations were based on a desired wall shear stress (WSS) of 0.5 dyn/cm², which has previously been shown to induce mucous secretions at normal physiological levels.

TABLE 1

| Number | Description | Target | Equation | Variable Definitions |
|---|---|---|---|---|
| Reynold's | Laminar vs. turbulent flow | <2300 | $\frac{UD}{{v}_{g}}$ | U = max flow velocity D = hydraulic diameter v_{g} = kinematic viscosity |
| | | | | of air |
| Womersley | Pulsatile flow | 1.0-1.68 | $\frac{D}{2} {( \frac{ω}{{v}_{g}} )}^{0.5}$ | D = hydraulic diameter ω = breathing frequency |
| | | | | v_{g} = kinematic viscosity |
| | | | | of air |
| Strouhal | Oscillatory flow | <1 | $\frac{ω D}{{u}_{avg}}$ | D = hydraulic diameter ω = breathing frequency |
| | | | | u_{avg} = average velocity |
| | | | | in nasal passage |

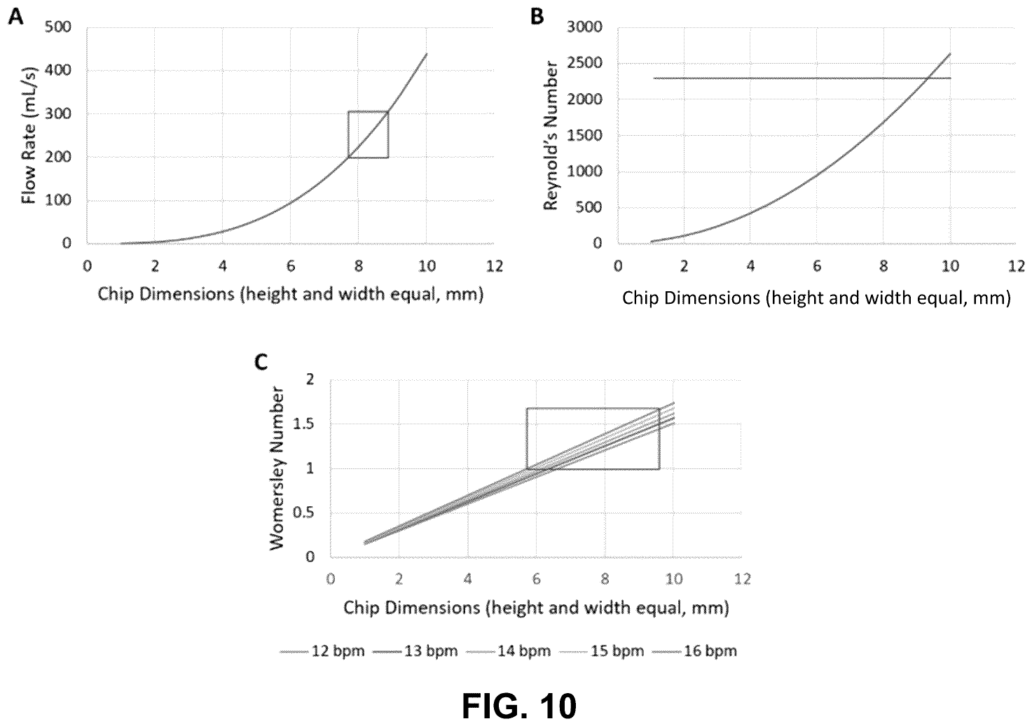

Based on the analysis shown in FIG. 10, an 8×8 mm cross section of the chip was selected so that physiologically relevant non-dimensional numbers and breathing rate could be achieved. With these dimensions, the Strouhal number is less than 1. Using these chip dimensions also allows for slight variations in velocity or breathing rate while still maintaining the requirements; however, any dimensions chosen within the boxed regions in FIG. 10 would be relevant for recapitulating the nasal airway with airflow at the targeted physiological conditions.

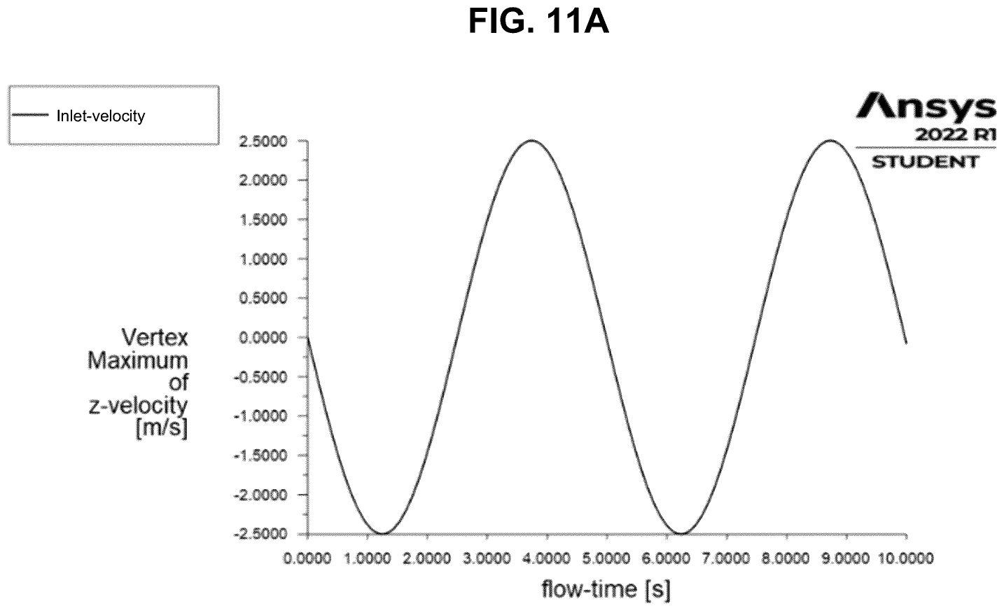

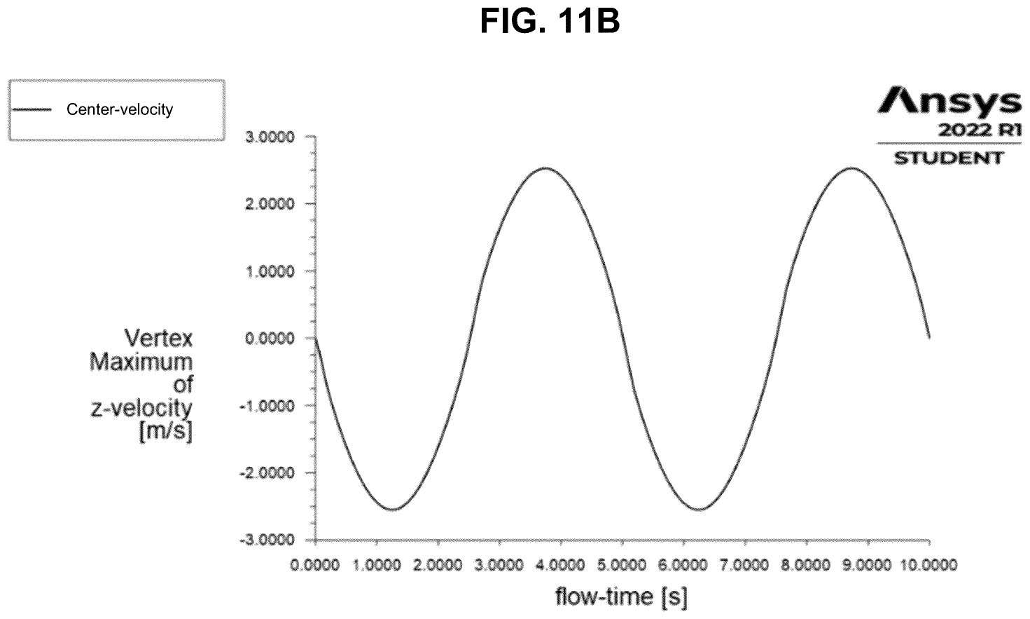

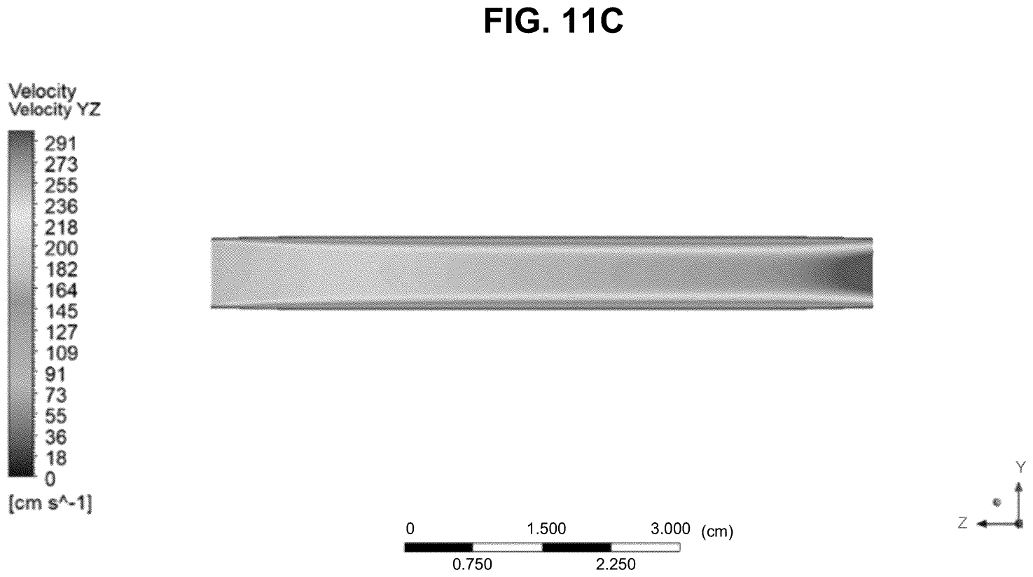

More in-depth computational fluid dynamics (CFD) simulations using ANSYS Fluent have been completed. These new simulations incorporate the shortened taper design, and also include Wall Shear Stress (WSS), an important parameter to consider when subjecting airway epithelial cells to airflow. In FIGS. 11A-11C, the velocity profile for physiological breathing is shown. A smooth parabolic velocity profile shows that the flow appears laminar through the chip.

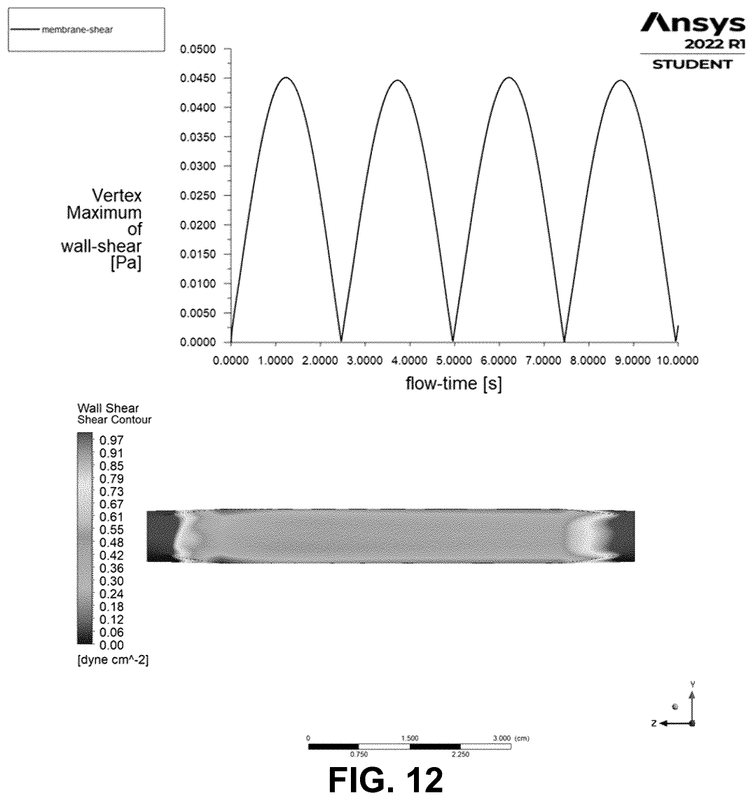

WSS at this flow rate was analyzed in the simulation and found to be approximately 0.45 dyne/cm² at the peak, which is within 10% of the target value (0.5 dyne/cm²). FIG. 12 shows the plot and profile of WSS.

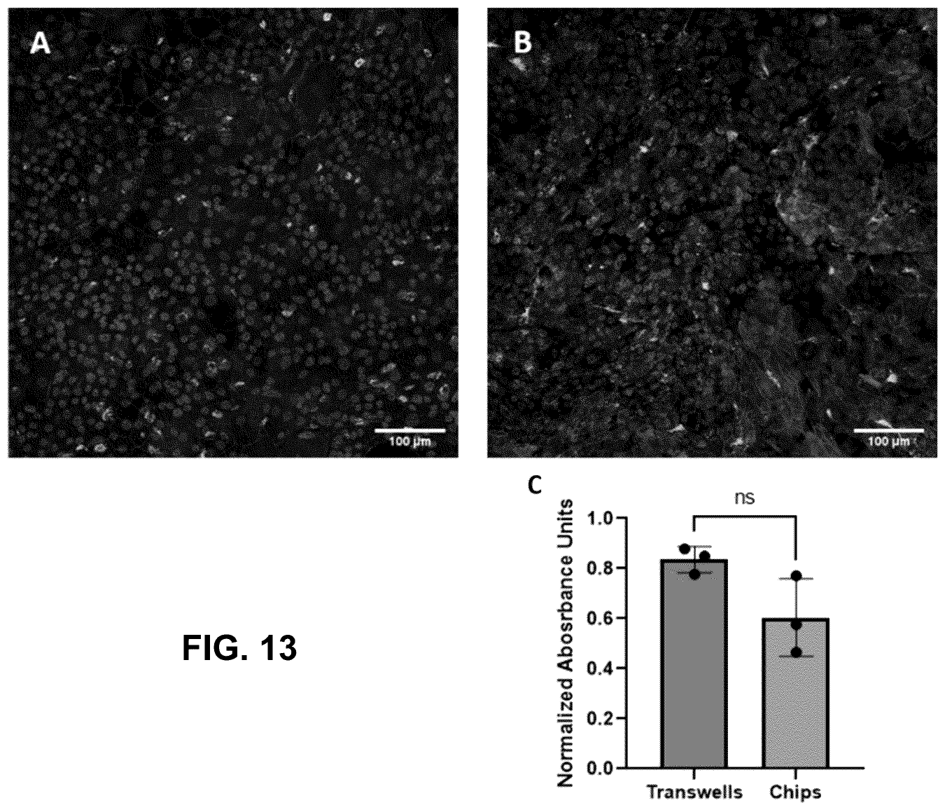

To ensure that cells can survive on the chip platform and exhibit normal nasal epithelial cell phenotypes, cells were cultured on chips and transwells for 14 days at the ALI and an MTS cell viability assay as well as immunostaining were performed. FIG. 13 shows ICC images of a confluent monolayer of cells on chips expressing aceylated tubulin (cilia, FIG. 13A) and MUC5AC (goblet cells, FIG. 13B). This data shows that, although the cells seem to have a slightly lower viability on chips, there is not a statistically significant difference in cell viability levels when cultured on chips versus transwells (FIG. 13C). More testing will be conducted to optimize cell viability on chips, but the preliminary findings suggest that cells are capable of surviving on the chip and expressing normal markers for a well-differentiated epithelial layer.

Physical Airflow Setup and Preliminary Flow Results

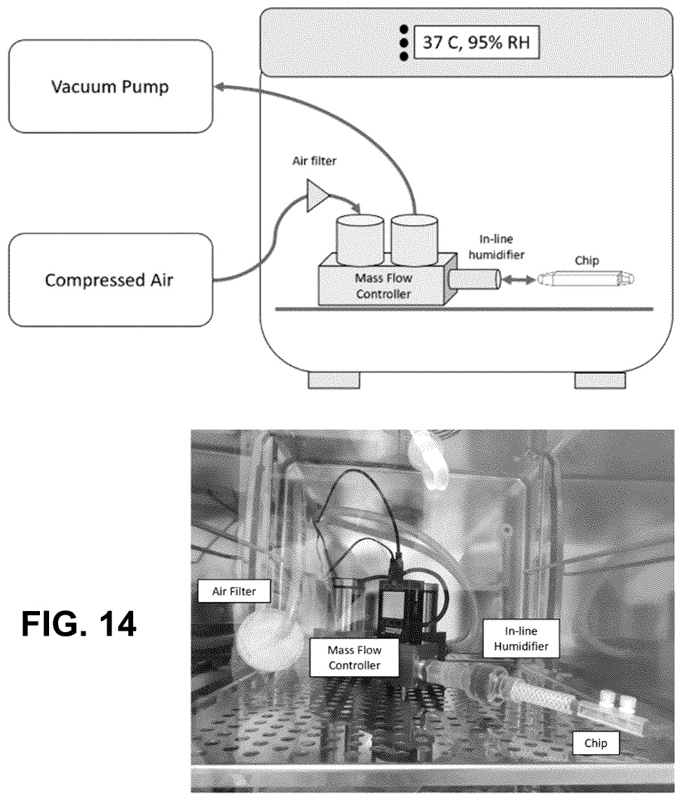

To test chips under airflow conditions, the setup shown in FIG. 14 was used. Briefly, a mass flow controller (Alicat Scientific) was connected to a filtered, compressed air source and a vacuum. An in-line humidifier was placed at the outlet of the controller just before the chip connection. The flow experiments are run in the cell culture incubator at 37° C. and 95% humidity.

The mass flow controller can be programmed to control air flow by inputting a sine wave function as previously described. For the preliminary flow experiments, the mass flow rate was set to 13.5 SLPM at a breathing rate of 12 cycles per minute in order to achieve a WSS of 0.5 dyne/cm² on the cells. Additional validations of flow rate and humidity levels are being conducted.

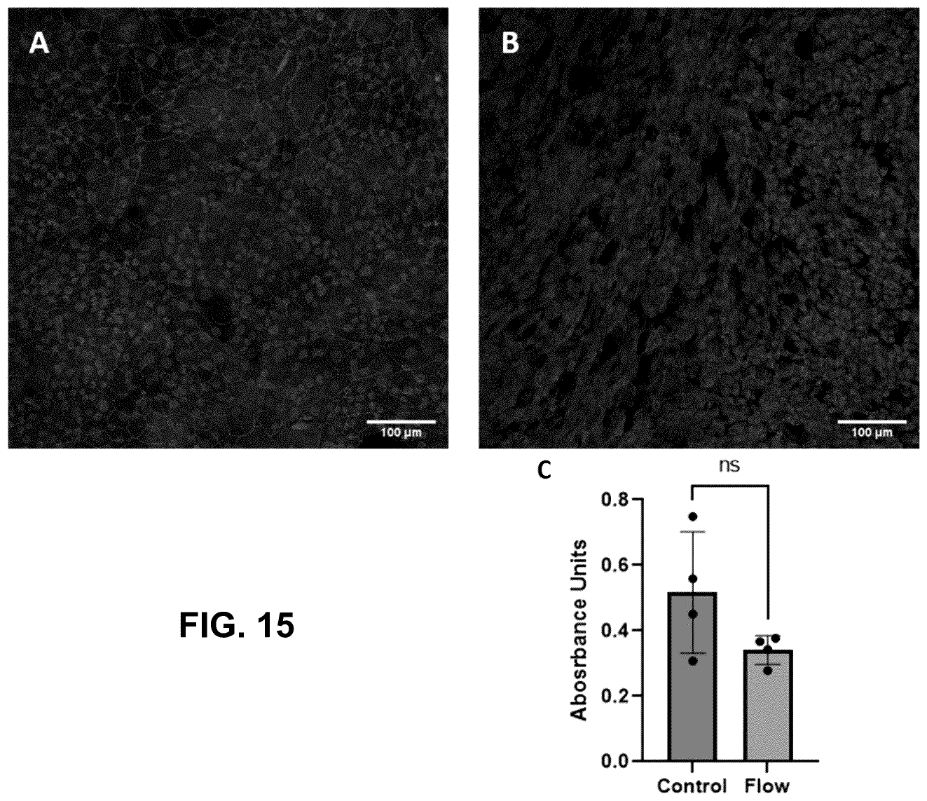

Preliminary flow experiments at 0.5 dyne/cm² are beginning to be conducted. Chips that had been in culture for 14 days at the ALI were exposed to oscillatory air flow for 30 minutes, then measured for cell viability and stained for ZO-1 to visualize any changes in the cell monolayer. The initial results, shown in FIG. 15, show some visual disparities between flow on chips (FIG. 15B) compared to control chips (no flow, FIG. 15A), with less expression of ZO-1. No statistical significance between viability of cells that were exposed to flow compared to the control was found (FIG. 15C). More experiments are being conducted to confirm these findings.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. An apparatus for simulating an airway, comprising:
an air channel having a central portion with an air inlet at a first end and an air outlet at a second end opposite the first end; and
a vascular channel adjacent to the central portion of the air channel, the vascular channel being separated from an interior of the central portion of the air channel by a porous membrane,
the air channel being configured to conduct air from the air inlet through the central portion such that air moves adjacent to the porous membrane.

2. The apparatus of claim 1, wherein the air channel comprises a flexible material.

3. The apparatus of claim 2, wherein a central portion of the air channel is enlarged and wherein the first end tapers down from the central portion to the air inlet and wherein the second end tapers down from the central portion to the air outlet.

4. The apparatus of claim 3, wherein the vascular channel comprises an inlet and an outlet for flow of cell culture media.

5. The apparatus of claim 4, wherein the porous membrane comprises a nano-porous membrane.

6. The apparatus of claim 5, wherein the porous membrane comprises a micro-porous membrane.

7. The apparatus of claim 6, wherein the porous membrane comprises a polycarbonate membrane.

8. The apparatus of claim 7, wherein a first side of the porous membrane facing the air channel comprises epithelial cells growing thereon, and
wherein a second side of the porous membrane opposite the first side and facing the vascular channel comprises microvascular endothelial cells growing thereon.

9. The apparatus of claim 8, further comprising fibroblasts growing on the porous membrane.

10. The apparatus of claim 9, further comprising:
a first electrode disposed within the air channel and in electrical communication with the porous membrane, and
a second electrode disposed within the vascular channel and in electrical communication with the porous membrane,
wherein the first electrode and the second electrode are configured to obtain at least one of a trans-epithelial electrical resistance or a trans-endothelial electrical resistance (TEER) across the porous membrane.

11. The apparatus of claim 10, further comprising an air pump coupled to the air channel,
wherein the air pump is configured to provide at least one of positive or negative air pressure to create continuous or cyclic air flow within the air channel.

12. The apparatus of claim 11, wherein the air pump is further configured to distribute particulate matter through the air channel.

13. The apparatus of any one of claims 1-12, further comprising a liquid pump coupled to the vascular channel,
wherein the liquid pump is configured to deliver cell culture media to the vascular channel.

14. The apparatus of claim 13, wherein the pump comprises a peristaltic pump. The apparatus of any one of claims 1-12, further comprising an in-line humidifier coupled to the air channel.

16. The apparatus of claim 1, wherein the central portion of the air channel comprises a nasal portion of the air channel,
wherein the vascular channel comprises a nasal vascular channel, and
wherein the porous membrane comprises a nasal porous membrane,
wherein the apparatus further comprises:
a lung portion coupled to the air outlet at the second end of the air channel,
a lung vascular channel adjacent to the lung portion,
wherein the lung vascular channel is separated from an interior of the
lung portion by a lung porous membrane, and
wherein the air channel is configured to conduct air from the air inlet
through the nasal portion to the lung portion.

17. The apparatus of claim 16, wherein the lung portion of the air channel comprises at least one branched passageway for air flow, and
wherein the lung vascular channel and the lung porous membrane each comprise at least one branched portion complementary to the at least one branched passageway of the lung portion of the air channel.

18. The apparatus of claim 17, wherein the lung portion of the air channel comprises a flexible material.

19. The apparatus of any one of claims 16-18, wherein a central portion of the nasal portion of the air channel is enlarged and wherein the first end tapers down from the central portion to the air inlet and wherein the second end tapers down from the central portion to the lung portion.

20. The apparatus of any one of claims 16-18, wherein the lung vascular channel comprises an inlet and an outlet for flow of cell culture media.

21. The apparatus of any one of claims 16-18, wherein the lung porous membrane comprises a nano-porous membrane.

22. The apparatus of any one of claims 16-18, wherein the lung porous membrane comprises a micro-porous membrane.

23. The apparatus of any one of claims 16-18, wherein the lung porous membrane comprises a polycarbonate membrane.

24. The apparatus of any one of claims 16-18, wherein a first side of the lung porous membrane facing the air channel comprises at least one of bronchial or tracheal epithelial cells growing thereon, and
wherein a second side of the lung porous membrane opposite the first side and facing the lung vascular channel comprises microvascular endothelial cells growing thereon.

25. The apparatus of any one of claims 16-18, further comprising fibroblasts growing on the lung porous membrane.

26. A method for simulating an airway, comprising:
providing an airway simulation apparatus comprising:
an air channel having a central portion with an air inlet at a first end and an air outlet at a second end opposite the first end, and
a vascular channel adjacent to the central portion of the air channel, the vascular channel being separated from an interior of the central portion of the air channel by a porous membrane; and
conducting, using the air channel, air from the air inlet through the central portion such that air moves adjacent to the porous membrane.

27. The method of claim 26, wherein providing an airway simulation apparatus further comprises:
providing the airway simulation apparatus wherein the air channel comprises a flexible material.

28. The method of claim 27, wherein providing an airway simulation apparatus further comprises:
providing the airway simulation apparatus wherein a central portion of the air channel is enlarged, wherein the first end tapers down from the central portion to the air inlet, and wherein the second end tapers down from the central portion to the air outlet.

29. The method of claim 28, wherein providing an airway simulation apparatus further comprises:
providing the airway simulation apparatus wherein the vascular channel comprises an inlet and an outlet for flow of cell culture media.

30. The method of claim 29, wherein providing an airway simulation apparatus further comprises:
providing the airway simulation apparatus wherein the porous membrane comprises a nano-porous membrane.

31. The method of claim 30, wherein providing an airway simulation apparatus further comprises:
providing the airway simulation apparatus wherein the porous membrane comprises a micro-porous membrane.

32. The method of claim 31, wherein providing an airway simulation apparatus further comprises:
providing the airway simulation apparatus wherein the porous membrane comprises a polycarbonate membrane.

33. The method of claim 32, wherein providing an airway simulation apparatus further comprises:
growing epithelial cells on a first side of the porous membrane facing the air channel, and
growing microvascular endothelial cells on a second side of the porous membrane opposite the first side and facing the vascular channel.

34. The method of claim 33, wherein providing an airway simulation apparatus further comprises:
growing fibroblasts on the porous membrane.

35. The method of claim 34, wherein providing an airway simulation apparatus further comprises:
disposing a first electrode within the air channel and in electrical communication with the porous membrane, and
disposing a second electrode within the vascular channel and in electrical communication with the porous membrane,
wherein the method further comprises:
obtaining, using the first electrode and the second electrode, at least one of a trans-epithelial electrical resistance or a trans-endothelial electrical resistance (TEER) across the porous membrane.

36. The method of claim 35, wherein providing an airway simulation apparatus further comprises:
coupling an air pump to the air channel,
wherein the method further comprises:
providing, using the air pump, at least one of positive or negative air pressure to create continuous or cyclic air flow within the air channel.

37. The method of claim 36, wherein providing at least one of positive or negative air pressure further comprises:
distributing, using the air pump, particulate matter through the air channel.

38. The method of any one of claims 26-37, further comprising a liquid pump coupled to the vascular channel,
wherein the liquid pump is configured to deliver cell culture media to the vascular channel.

39. The method of claim 38, wherein providing an airway simulation apparatus further comprises:
providing the pump wherein the pump comprises a peristaltic pump.

40. The method of any one of claims 26-37, further comprising an in-line humidifier coupled to the air channel.

41. The method of claim 26, wherein providing an airway simulation apparatus further comprises:
providing the airway simulation apparatus wherein the central portion of the air channel comprises a nasal portion of the air channel, wherein the vascular channel comprises a nasal vascular channel, and wherein the porous membrane comprises a nasal porous membrane,
wherein the method further comprises:
coupling a lung portion to the air outlet at the second end of the air channel,
providing a lung vascular channel adjacent to the lung portion, wherein the lung vascular channel is separated from an interior of the lung portion by a lung porous membrane, and
conducting air, using the air channel, from the air inlet through the nasal portion to the lung portion.

42. The method of claim 41, wherein providing an airway simulation apparatus further comprises:
providing the airway simulation apparatus wherein the lung portion of the air channel comprises at least one branched passageway for air flow, and wherein the lung vascular channel and the lung porous membrane each comprise at least one branched portion complementary to the at least one branched passageway of the lung portion of the air channel.

43. The method of claim 42, wherein providing an airway simulation apparatus further comprises:
providing the airway simulation apparatus wherein the lung portion of the air channel comprises a flexible material.

44. The method of any one of claims 41-43, wherein providing an airway simulation apparatus further comprises:
providing the airway simulation apparatus wherein a central portion of the nasal portion of the air channel is enlarged, wherein the first end tapers down from the central portion to the air inlet, and wherein the second end tapers down from the central portion to the lung portion.

45. The method of any one of claims 41-43, wherein providing an airway simulation apparatus further comprises:
providing the airway simulation apparatus wherein the lung vascular channel comprises an inlet and an outlet for flow of cell culture media.

46. The method of any one of claims 41-43, wherein providing an airway simulation apparatus further comprises:
providing the airway simulation apparatus wherein the lung porous membrane comprises a nano-porous membrane.

47. The method of any one of claims 41-43, wherein providing an airway simulation apparatus further comprises:
providing the airway simulation apparatus wherein the lung porous membrane comprises a micro-porous membrane.

48. The method of any one of claims 41-43, wherein providing an airway simulation apparatus further comprises:
providing the airway simulation apparatus wherein the lung porous membrane comprises a polycarbonate membrane.

49. The method of any one of claims 41-43, wherein providing an airway simulation apparatus further comprises:
growing at least one of bronchial or tracheal epithelial cells on a first side of the lung porous membrane facing the air channel, and
growing microvascular endothelial cells on a second side of the lung porous membrane opposite the first side and facing the lung vascular channel.

50. The method of any one of claims 41-43, wherein providing an airway simulation apparatus further comprises:
growing fibroblasts on the lung porous membrane.
